(19) United States
(12) Patent Application Publication
(10) Pub. No.: US 2020/0155703 A1
(43) Pub. Date: May 21, 2020

(54) Therapeutic Membrane Vesicles

(71) Applicant: Codiak BioSciences, Inc., Cambridge, MA (US)

(72) Inventors: Jan Lõtvall, Boston, MA (US); Su Chul Jang, Pohang (KR)

(21) Appl. No.: 16/084,169

(22) PCT Filed: Mar. 15, 2017
(86) PCT No.: PCT/US2017/022544

§ 371 Date: Sep. 11, 2018

Related U.S. Application Data
(60) Provisional application No. 62/308,805, filed on Mar. 15, 2016.

(51) Int. Cl. A61K 47/69; A61K 38/18; C07K 16/28; C12N 15/10; A61K 9/127; C12N 15/113; C12N 15/90; C12N 9/22

(52) U.S. Cl. CPC A61K 47/6913; A61K 38/1841; C07K 16/2896; C12N 15/102; C12N 2310/20; C12N 15/113; C12N 15/907; C12N 9/22; A61K 9/1278

(57) ABSTRACT

The present invention relates to a method for producing membrane vesicles from extracellular vesicles or organelles and therapeutic membrane vesicles produced by such method. The invention further relates to therapeutic membrane vesicles, a method of treating a metabolic disorder by using such vesicles and such vesicles for use in therapy, such as in treatment of a metabolic disorder. The invention further relates to a method of producing a membrane vesicle from an organelle. In addition, the present invention relates to a method of separating a sub-population of extracellular vesicles from an extracellular vesicle bulk.

### TECHNICAL FIELD

The present invention relates to the field of production of membrane vesicles, in particular production of therapeutic membrane vesicles. Moreover, the present invention relates to therapeutic use of such membrane vesicles for targeted delivery of therapeutic compounds.

### BACKGROUND OF THE INVENTION

Extracellular vesicles such as exosomes, ectosomes, microvesicles and apoptotic bodies are known to be released by many cells in the human body, and can shuttle functional RNA molecules as well as proteins to other cells. The cargo of these extracellular vesicles is protected from extracellular enzymes and the immune system by a lipid membrane bilayer. It has previously been suggested that extracellular vesicles such as exosomes can be utilized for the delivery of functional molecules, including therapeutic nucleotides to diseased cells, such as cancer cells, cancerous tissues and inflammatory cells.

Several technologies have been proposed for loading of exosomes or microvesicles with for example therapeutic RNA cargo for delivery to the inside of a recipient cell (Alvarez-Erviti L. et al., Nat Biotechnol. 2011 April; 29(4):341-5; Ohno S. et al., Mol Ther. 2013 January; 21(1):185-91: EP2010663). Common to all these technologies is that they utilize relatively “intact” vesicles. The vesicles can be considered intact in that they carry the molecules they normally carry, apart from any specific cargo they have been loaded with, for example siRNAs or microRNAs.

Although much work has previously been done, the field of extracellular vesicles is not yet fully explored. The full therapeutic potential of such vesicles still remains to be realized.

### SUMMARY OF THE INVENTION

It is an object of the present disclosure to provide new therapeutic membrane vesicles which can be used for targeted delivery of therapeutic compounds. A further object is use of the thus provided vesicles in therapy.

It is an object of the present disclosure to provide new methods for producing membrane vesicles, including those for therapeutic use. Such methods include inter alia methods for isolating specific membrane vesicles and for providing membrane vesicles with a pre-determined molecular content.

These and other objects, which are evident to the skilled person from the present disclosure, are met by the different aspects of the invention as claimed in the appended claims and as generally disclosed herein.

As used herein, the term “extracellular vesicle” refers to a cell-derived vesicle comprising a membrane that encloses an internal space. Extracellular vesicles comprise all membrane-bound vesicles that have a smaller diameter than the cell from which they are derived. Generally extracellular vesicles range in diameter from 20 nm to 1000 nm, and can comprise various macromolecular cargo either within the internal space, displayed on the external surface of the extracellular vesicle, and/or spanning the membrane. Said cargo can comprise nucleic acids, proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, and/or cultured cells.

As used herein, the term “nanovesicle” refers to a cell-derived small (between 20-250 nm in diameter, more preferably 30-150 nm in diameter) vesicle comprising a membrane that encloses an internal space, and which is generated from said cell by direct or indirect manipulation such that said nanovesicle would not be produced by said producer cell without said manipulation. Appropriate manipulations of said producer cell include but are not limited to serial extrusion, treatment with alkaline solutions, sonication, or combinations thereof. The production of nanovesicles can, in some instances, result in the destruction of said producer cell. Preferably, populations of nanovesicles are substantially free of vesicles that are derived from producer cells by way of direct budding from the plasma membrane or fusion of the late endosome with the plasma membrane. The nanovesicle comprises lipid or fatty acid and polypeptide, and optionally comprises a payload (e.g. a therapeutic agent), a receiver (e.g. a targeting moiety), a polynucleotide (e.g. a nucleic acid, RNA, or DNA), a sugar (e.g. a simple sugar, polysaccharide, or glycan) or other molecules. The nanovesicle, once it is derived from a producer cell according to said manipulation, can be isolated from the producer cell based on its size, density, biochemical parameters, or a combination thereof. Unless otherwise specified, the term “membrane vesicle” or “therapeutic membrane vesicle” refers to a type of nanovesicle.

As used herein, the term “exosome” refers to a cell-derived small (between 20-300 nm in diameter, more preferably 40-200 nm in diameter) vesicle comprising a membrane that encloses an internal space, and which is generated from said cell by direct plasma membrane budding or by fusion of the late endosome with the plasma membrane. Generally, production of exosomes does not result in the destruction of the producer cell. The exosome comprises lipid or fatty acid and polypeptide, and optionally comprises a payload (e.g. a therapeutic agent), a receiver (e.g. a targeting moiety), a polynucleotide (e.g. a nucleic acid, RNA, or DNA), a sugar (e.g. a simple sugar, polysaccharide, or glycan) or other molecules. The exosome can be derived from a producer cell, and isolated from the producer cell based on its size, density, biochemical parameters, or a combination thereof.

As used herein, the term “organelle” means a specialized subunit within a cell that has a specific function. Individual organelles are usually separately enclosed within their own lipid bilayers, i.e. membranes. Non-limiting, exemplary organelles include chloroplasts, the endoplasmic reticulum, flagellum, Golgi apparatus, mitochondria, endosome, lysosome, vacuole and the nucleus.

As used herein, the term “epitope specific binder” means a molecule that binds to a specific epitope. An epitope is the part of an antigen that is recognized by the immune system, specifically by antibodies, B cells, or T cells, phage, or aptamers. An “epitope specific binder” can or cannot be further bound to, for example, a surface of a bead. Examples of epitope specific binders include antibodies, B cells, or T cells, or aptamers.

As used herein, the term “membrane” means biological membranes, i.e. the outer coverings of cells and organelles that allow passage of certain compounds. In some contexts, the term “membrane” can refer to a lipid bilayer that at one time bounded an extracellular vesicle or organelle and enclosed an intravesicular or organellar content and that subsequently was opened to expose the interior contents of the extracellular vesicle or organelle and dissociate those contents from the opened membrane.

According to one aspect, a method is provided for producing membrane vesicles comprising:
a. providing extracellular vesicles or organelles;
b. opening said extracellular vesicles or said organelles by treatment with an aqueous solution having a pH of from 9 to 14 to obtain membranes;
c. removing intravesicular or organellar content; and
d. re-assembling said membranes to form membrane vesicles.

The above-defined method provides membrane vesicles, produced from extracellular vesicles or organelles, that have been opened, released from their intravesicular or organellar content, and then reassembled. Membrane vesicles produced in this way are devoid of detrimental cargo that they can naturally contain, including harmful endogenous molecules such as DNA or nuclear membrane components, or any unwanted RNA species, enzymes or other proteins, as well as infectious components such as viruses, virus components including virus genomic material and or prions or similar infectious constituents. Removal of any naturally-occurring intravesicular content from extracellular vesicles reduces possible side-effects caused by such intravesicular content, and thus reduces the risk of unwanted effects. Similarly, removal of any naturally-occurring organellar content from organelles reduces possible side-effects caused by such organellar content, and thus reduces the risk of unwanted effects.

By removal of any naturally-occurring content, the membrane vesicles can be loaded with a therapeutic compound and thereby be used to induce a pure therapeutic effect. While avoiding any potential negative side-effects that an intravesicular or organellar content can provide, the effect of the surface molecules is maintained. Removal of any inner content in this way will preferably not affect the function of the membrane bound/surface bound molecules. These are preferably maintained, hence their function are preferably maintained. The function of such membrane/surface molecules can be a targeting function or a therapeutic function.

The therapeutic membrane vesicles as disclosed herein potentially solve multiple problems with current extracellular vesicle therapeutics, by for example optimizing yield of membrane vesicles (compared with extracellular vesicles).

According to one embodiment of this aspect, the method is limited to extracellular vesicles.

According to one embodiment of this aspect, the method is limited to organelles.

In some embodiments, step d of the method described herein can be done by one or more of sonication, mechanical vibration, extrusion through porous membranes, electric current and combinations thereof. Re-assembling membranes of opened extracellular vesicles or organelles can accordingly be accomplished by sonication, mechanical vibration, extrusion through porous membranes, electric current or combinations thereof. One or more of these techniques can be employed.

In some embodiments, step d of the method described herein can be done by sonication. Typically, the reassembly of membranes of opened extracellular vesicles or organelles is done by sonication.

Said removing of intravesicular or organellar cargo molecules of step c of the method described herein can be done by ultracentrifugation or density gradient ultracentrifugation.

In some aspects, the method further comprises:
e. loading a cargo into said membrane vesicles, wherein step e can be performed concomitantly with or after step d.

Concomitantly with or after the step of re-assembling emptied vesicles or organelles, the newly formed membrane vesicles can be loaded with very specific cargos, including different types of synthetic molecules and/or proteins or polypeptides with intracellular or extracellular targets, or nucleotides that can influence the cell function, phenotype, proliferation or viability, or proteins, peptides or hormones with similar function. Proteins or polypeptides with intracellular or extracellular targets include bioactive or inhibitory polypeptides such as hormones, cytokines, chemokines, receptors, and enzymes. Further examples of cargo are defined below.

In some embodiments, step e of the method described herein can be done by physical manipulation after step d, wherein said physical manipulation is selected from electroporation, sonication, mechanical vibration, extrusion through porous membranes, electric current and combinations thereof. Loading of cargo to vesicles formed by membranes from opened extracellular vesicles or organelles can be done by mixing, co-incubation, electroporation, sonication, mechanical vibration, extrusion through porous membranes, electric current and combinations thereof after the reassembly of such membranes.

In some embodiments, said loading of step e can be done concomitantly with step d. A cargo of specific molecules is, for example, mixed with the opened (membrane) form of the extracellular vesicles or organelles, followed by reassmembly to form a membrane vesicle using e.g. any one of the above defined methods.

Said cargo can be selected from a synthetic bioactive compound, a natural bioactive compound, an antibacterial compound, an antiviral compound, a protein or a polypeptide, a nucleotide, a genome editing system, microRNA, siRNA, long-non-coding RNA, antago-miRs, morpholino, mRNA, t-RNA, y-RNA, RNA mimics, DNA, and combinations thereof. Cargo loaded to into membrane vesicles concomitantly or after the reassembly of the membranes can be of many specific types, such as RNA-interference molecules (RNAi: microRNA or siRNA or long-non-coding RNA, antago-miRs, morpholino or any other molecules that can have RNA-interference function or that can block RNA or protein function in the cell, including transcription factors), mRNA (messenger RNA in full or reduced length to produce a functional protein in a recipient cell), t-RNA, y-RNA, RNA mimics, DNA molecules (to deliver either functional short DNA probes or whole DNA gene sequences to replace or repress dysfunction in the recipient cell) enzyme inhibitors or other small molecule drugs. It can also be natural or synthetic hormones, to e.g. optimize intracellular delivery, as well as synthetic small molecules with pharmacological function within the cell cytoplasm or cell organelles or cell nucleus. It is contemplated that one or more of these specific types of molecules can be loaded into the membrane vesicles, either during their formation or after their formation.

Said cargo can be a compound related to anti-inflammatory function, pro-inflammatory function, or cell migration. In one embodiment, said cargo is TGF-beta.

Said cargo can be a genome editing system. The genome editing system includes, without limitation, a meganuclease system, a zinc finger nuclease (ZFN) system, a transcription activator-like effector nuclease (TALEN) system, and a clustered regularly interspaced short palindromic repeats (CRISPR) system. The CRISPR system can be a CRISPR-Cas9 system. The CRISPR-Cas9 system comprises a nucleotide sequence encoding a Cas9 protein, a nucleotide sequence encoding a CRISPR RNA that hybridizes with the target sequence (crRNA), and a nucleotide sequence encoding a trans-activating CRISPR RNA (tracrRNA). The crRNA and the tracrRNA can be fused into one guide RNA. The components of the CRISPR-Cas system can be located in the same vector or in different vectors. The CRISPR-Cas9 system can further comprise a nuclear localization signal (NLS).

Said cargo can be a microRNA or an siRNA that specifically binds to a transcript encoding a mutated or non-mutated oncogene. The binding of the microRNA or the siRNA can inhibit the mRNA translation and protein synthesis of oncogenes. Such oncogenes include, but are not limited to, ABLI, BLC1, BCL6, CBFA1, CBL, CSFIR, ERBA, ERBB, EBRB2, ETS1, ETS1, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, and YES. In one embodiment, the oncogene is a mutated KRAS, for example KRAS G12D, KRAS G12C, or KRAS G12V. In another embodiment, the oncogene is a Myc, such as N-Myc, c-Myc, or L-Myc.

The therapeutic membrane vesicles can have at least one physiological property different from the population of extracellular vesicles or organelles from which said membrane vesicles derive, wherein the physiological property is related to one or more of biodistribution, cellular uptake, half-life, pharmacodynamics, potency, dosing, immune response, loading efficiency, stability, or reactivity to other compounds. The different physiological property can be measured by various methods known in the art. The different physiological property can be improved targeting efficiency, improved delivery, or an increase in therapeutic cargo to a recipient cell, organ, or subject. The cargo can be loaded into the membrane vesicle more efficiently than the cargo is loaded into extracelluar vesicles or organelles from which said membrane vesicles are derived.

Said extracellular vesicles can be a sub-population of extracellular vesicles derived from an extracellular vesicle bulk, or wherein said organelles are one sub-type of organelle derived from a plurality of organelles.

The released vesicles from any cell, or from any tissue, include a cloud of vesicles with different content, different surface molecules and in some cases from different cellular origin. A sub-population of extracellular vesicles can have very specific characteristics with regard to for example surface molecules, functions and targets. Such extracellular vesicles can originate from the cell membrane, the Golgi-apparatus, the endoplasmic reticulum, the nucleus or mitochondria.

Similarly, specific sub-types of organelles can be derived from the plurality of organelle types present in a cell. For example, a plurality of organelles can be removed from a cell lysate by conventional techniques such as density gradient approaches. One or more specific sub-types of organelles can subsequently be isolated, e.g. by employment of techniques as disclosed herein. Specific sub-types of organelles which are contemplated for use in the present method include the Golgi-apparatus, the endoplasmic reticulum, the lysosome, the endosome, the nucleus or mitochondria.

In some aspects, the method comprises prior to step a:

contacting an epitope specific binder with said extracellular vesicle bulk or said

organelles; and separating a sub-population of extracellular vesicles or sub-type of

organelles from said extracellular vesicle bulk or plurality of organelles.

As each subgroup of vesicles can carry very distinct molecules both on their surface as well as intravesicular cargo, they can be separated by positive isolation and/or negative isolation from other vesicles by epitope specific binding techniques. Typically, epitope specific binders for isolation can be one or more of a specific antibody, phage or an aptamer. Separation of organelles or vesicles of different sub-cellular origin from each other can provide purified extracellular vesicles or organelles having specific characteristics. Such specific characteristics can include a desirable molecular cargo as mentioned above, and/or an ability to be taken up by targeted cells for delivery of for example surface molecules and/or cargo molecules to said targeted cells. An epitope specific binder can also be chosen such that an unwanted sub-population is bound, which sub-population can thus be removed from the extracellular bulk, by so called negative isolation.

Said epitope specific binder can be an antibody against at least one mitochondrial membrane protein, preferably MTCO2 protein. By using an antibody against a mitochondrial membrane protein, preferably MTCO2, as the epitope specific binder, the isolated sub-population of extracellular vesicles have increased ATP synthase activity.

Said epitope specific binder can be an antibody against the surface marker CD63. By using an antibody against CD63 as the epitope specific binder, the isolated sub-population of extracellular vesicles can have reduced RNA content as compared to the extracellular vesicle bulk.

Included within the scope of this disclosure are therapeutic membrane vesicles comprising:
vesicles formed from membranes, said membranes being derived from extracellular vesicles or organelles.

The therapeutic membrane vesicles as disclosed herein solve multiple problems with current extracellular vesicle therapeutics, by for example improving targeting efficacy to a recipient cell, organ or object. The therapeutic membrane vesicles mimic extracellular vesicles, such as exosomes, by their ability to interact with a recipient cell via their surface molecules. It is to be understood that the therapeutic membrane vesicles are formed from membranes derived from extracellular vesicles or organelles. For instance, said membranes can be derived from extracellular vesicles or organelles by opening of such extracellular vesicles or organelles. The resulting emptied therapeutic membrane vesicles can thus be devoid of detrimental content that natural extracellular vesicles or organelles contain, including harmful endogenous molecules such as DNA or nuclear membrane components, or any unwanted RNA species, enzymes or other proteins, as well as infectious components such as viruses, virus components including virus genomic material and or prions or similar infectious constituents.

The therapeutic membrane vesicles can be loaded with a therapeutic cargo. Therapeutic membrane vesicles comprising a loaded cargo mimic naturally-occurring extracellular vesicles in that they have an ability to interact with a recipient cell via their surface molecules, and to deliver their cargo to said recipient cell. Examples of therapeutic cargo that can be contained within a therapeutic membrane vesicle according to this aspect are disclosed elsewhere herein, but can typically include one or more of a synthetic bioactive compound, an antibacterial compound, an antiviral compound, a natural bioactive compound, a protein, a nucleotide, a genome editing system, microRNA, siRNA, long-non-coding RNA, antagomiRs, morpholino, mRNA, t-RNA, y-RNA, RNA mimics, DNA, and combinations thereof.

Said therapeutic cargo can comprise an enzyme that catalyzes the production of ATP. Therapeutic membrane vesicles comprising an enzyme that catalyzes the production of ATP, such as ATP synthase, can have beneficial effects on metabolic conditions.

Said therapeutic cargo can comprise a compound with the capability of influencing the phenotype and/or function of mesenchymal stem cells such as to increase the anti-inflammatory function of said mesenchymal stem cells. Contacting mesenchymal stem cells with therapeutic membrane vesicles comprising TGF-beta can increase the migratory activity, the wound healing activity, and the therapeutic efficiency of such stem cells. Exposing mesenchymal stem cells to therapeutic membrane vesicles comprising a compound with the capability of influencing the phenotype and/or function of such stem cells can accordingly increase the anti-inflammatory function of such stem cells. For example, such therapeutic membrane vesicles can reduce the inflammation in a mouse model of asthma.

Said therapeutic cargo can comprise a compound related to anti-inflammatory function, or a compound related to pro-inflammatory function. Examples of compounds related to anti-inflammatory function include IL-10, interferon alfa, interferon gamma and an anti-inflammatory microRNA. Said anti-inflammatory microRNA is for example miR-146. Examples of compounds related to pro-inflammatory function include TGF-beta, TNF-alfa, IL-4, IL-6, a toll-like receptor ligand and a pro-inflammatory microRNA. Said pro-inflammatory microRNA is for example miR-10, -29, or -155).

The cargo can comprise a genome editing system, such as a CRISPR-Cas9 system. The therapeutic membrane vesicles loaded with a CRISPR-Cas9 system can be used to alter gene expression and function for disease treatment, regenerative medicine, and tissue engineering.

The cargo can be loaded into the membrane vesicles more efficiently than the cargo is loaded into extracelluar vesicles or organelles from which said membrane vesicles are derived. Removal of any naturally-occurring content, such as unwanted RNA species, enzymes or other proteins, as well as infectious components such as viruses, virus components including virus genomic material and/or prions or similar infectious constituents, of the membrane vesicle can avoid contamination or harm to the therapeutic cargo.

Said extracellular vesicles can represent a sub-population of an extracellular vesicle bulk having a different sub-set of membrane and surface molecules than the extracellular vesicle bulk. A sub-population of an extracellular vesicle bulk having a different sub-set of membrane and surface molecules than the extracellular vesicle bulk can have a more specific effect and thus fewer side effects. Examples of sub-populations of an extracellular vesicle bulk are disclosed elsewhere herein. It is contemplated that membrane vesicles derived from a specific sub-population of extracellular vesicles can be particularly useful, e.g. due to the presence of specific membrane and/or surface molecules, or for targeted delivery of a specific therapeutic cargo.

In some aspects, organelles described herein represent a sub-type of organelles derived from a plurality of organelles. Examples of sub-types of organelles derived from a plurality of organelles are disclosed elsewhere herein. It is contemplated that membrane vesicles derived from a specific sub-type of organelles can be particularly useful, e.g. due to the presence of specific membrane and/or surface molecules, for targeted delivery of a specific therapeutic cargo.

Membrane vesicles can be characterized by at least one of
i. surface membrane molecules are inverted;
ii. at least one type of surface molecules with the capability of influencing the phenotype and function of mesenchymal stem cells such that the anti-inflammatory function of said mesenchymal stem cells is increased;
iii. at least one type of surface marker common to extracellular vesicles is either present or absent;
iv. at least one type of mitochondrial membrane surface molecule is present;
v. at least one type of nuclear membrane surface molecule is present: and
vi. at least one type of membrane molecule from Golgi and/or Endoplasmic reticulum is present.

Therapeutic membrane vesicles having their surface membrane molecules inverted can allow them to directly deliver surface molecules with second messages, such as intracellular signaling. Further, therapeutic membrane vesicles having surface molecules with the capability of influencing the phenotype and/or function of mesenchymal stem cells can increase the anti-inflammatory function of the mesenchymal stem cells.

With reference to Example 4 and the common surface marker CD63, CD63 negative extracellular vesicles contain much RNA, whereas CD63 positive extracellular vesicles are devoid of RNA, which suggests that each sub-population of extracellular vesicles has different cargo and thus the ability to potentially induce a specific effect.

With reference to Example 3, the presence of one type of mitochondrial membrane surface molecule, MTCO2, on a membrane vesicle gave a higher ATP synthase activity. It is thus assumed that membrane vesicles of other organellar origin such as nuclear, Golgi or endoplasmic reticulum origin, in a similar way display other properties or functions depending on their different membrane surface molecules. Therapeutic membrane vesicles having any one of the specific characteristics as set out above can further be derived from a particular sub-population of extracellular vesicles or a particular sub-type of organelles.

In some aspects as disclosed herein, said therapeutic membrane vesicles have a capability of migrating through tissues. It has been shown that in certain instances, the therapeutic membrane vesicles have improved motility, as they can change their shape when still not fixed. This results in visible shape-changes of cell-free vesicles in vitro. This is related to the presence in certain sub-population/sub-type of vesicles, of the motility protein, actin, and associated proteins, the presence of which can be determined using vesicular proteomics approaches.

The therapeutic membrane vesicles can have increased motility as compared to the extracellular vesicles or organelles from which said membrane vesicles are derived.

Extracellular vesicles or organelles can originate from a cancer cell, a cancer cell line, an inflammatory cell, a structural cell, a neural/glial cell/oligodendrocyte or a mesenchymal/embryonic stem cell.

Extracellular vesicles or organelles can be isolated from a normal or diseased tissue, including a tumor, bone marrow, or immune cells isolated from blood, lymph nodes or spleen.

Membrane vesicles can be obtained by a method as defined in any one of the aspects as disclosed herein.

Therapeutic membrane vesicles according to the aspects as disclosed herein can be used in therapy. Therapeutic membrane vesicles according to the invention can solve multiple problems with current extracellular vesicle therapeutics, by e.g. improving targeting efficacy, delivery, and increasing therapeutic cargo to a recipient cell/organ/object. Therapeutic membrane-vesicles could be said to mimic exosomes or any other extracellular vesicle by mimicking the latter in their ability to interact with a recipient cell via their surface molecules, and/or to deliver a therapeutic cargo to a recipient cell. At the same time, the therapeutic membrane vesicles differ from exosomes and other naturally-occurring extracellular vesicles in that the former can mitigate possible contamination with unwanted extracellular vesicles with possible negative side effects, as well as any detrimental content these can naturally contain. Moreover, the therapeutic membrane-vesicles can also be loaded with antibiotics or antiviral molecules, to treat intracellular infections such as intracellular bacteria, viruses or prions, including Epstein-Barr virus, HIV or any other infectious species.

Therapeutic membrane vesicles can be used in treatment of a metabolic disorder. The therapeutic membrane vesicles can have the capacity to deliver enzymes important for the production of ATP, such as the enzyme ATP synthase.

Therapeutic membrane vesicles can be used in a method of treating a disorder comprising administering therapeutic membrane vesicles according to the invention to a patient in need thereof.

Therapeutic membrane vesicles can be used in a method of treating a metabolic disorder comprising administering therapeutic membrane vesicles according to the invention, to a patient in need thereof.

Therapeutic membrane vesicles can be used for targeted delivery of said therapeutic cargo. Therapeutic membrane vesicles produced from a sub-population of extracellular vesicles or organelles can have specific surface molecules that enable them to reach specific targets and deliver their therapeutic cargo, leading to a more specific treatment as well as the delivery of specific therapeutic cargo. Therapeutic cargo can for example target intracellular functions such as mutated or non-mutated oncogenes in malignant disease, or any other intracellular process or function including transcription factors, protein production, hormone receptors, cytokines, membrane folding, energy production, proliferation, DNA replication, or any other intracellular function. The therapeutic membrane vesicles can also be loaded with antibiotics or antiviral molecules, to treat intracellular infections such as intracellular bacteria, viruses or prions, including Epstein-Barr virus, HIV or any other infectious species.

Included in the scope of this disclosure is a method of producing a membrane vesicle from an organelle comprising:
a. lysing a cell to release cellular content;
b. separating said organelle from said cellular content; and
c. opening said organelle by treating said organelle with an aqueous solution with pH 9-14 to obtain a membrane; and
d. re-assembling said membrane to form a membrane vesicle.

Organelles that have been emptied of their content will be devoid of detrimental cargo that they can naturally contain, including harmful endogenous molecules such as DNA or nuclear membrane components, or any unwanted RNA species, enzymes or other proteins, as well as infectious components such as viruses, virus components including virus genomic material and or prions or similar infectious constituents. Removing the content from organelles can reduce possible side-effects caused by such content and thus decrease possible unwanted effects.

Said organelle can be a mitochondrion.

The method can further comprise:
c. loading a cargo to said membrane vesicle, wherein step e can be performed concomitantly with or after step d.

Concomitantly with or after the process of re-assembling emptied organelles, the newly formed vesicles can be loaded with specific cargos, including different types of synthetic molecules/chemicals and or proteins (including bioactive or inhibitory molecules such as hormones, cytokines, chemokines, receptors, or enzymes) with intracellular or extracellular targets, or nucleotides that can influence the cell function, phenotype, proliferation or viability, or proteins, peptides or hormones with similar function. Other examples of cargo that can be incorporated into the membrane vesicles are disclosed in connection with other aspects. Membrane vesicles produced according to this aspect can be useful in therapy, e.g. for targeted delivery of a particular therapeutic cargo.

Included in the scope of this disclosure is a method of producing membrane vesicles from organelles comprising:
a. opening a cell by treating said cell with an aqueous solution with pH 9-14 to obtain a mixture of membranes;
b. separating organellar membranes from said mixture of membranes, and
c. re-assembling said organellar membranes to form membrane vesicles.

The method can further comprise:
d. loading a cargo to said membrane vesicles, wherein step d can be performed concomitantly with or after step c.

Examples of cargo that can be loaded into the membrane vesicles are disclosed elsewhere herein. Membrane vesicles obtained by this method can preferably be used in therapy. Likewise, exemplary methods for separating organellar membranes from mixtures are disclosed herein, e.g. by use of an epitope specific binder. In one embodiment, said separation further comprises separation of one or more sub-types of organelles from said mixture.

Included in the scope of this disclosure is a method of separating a sub-population of extracellular vesicles from an extracellular vesicle bulk, comprising:
contacting an epitope specific binder with said extracellular vesicle bulk; and
separating said sub-population of extracellular vesicles from said extracellular vesicle bulk.

The released vesicles from any cell, or from any tissue, include a cloud of vesicles with different content, surface molecules and with cellular origin. A sub-population of extracellular vesicles can have very specific characteristics and have less diversity with regard to for example surface molecules, functions and targets. A subpopulation of extracellular vesicles can be separated by positive and/or negative separation with epitope specific binders. Typically, the epitope specific binder can be an antibody, a phage, or an aptamer.

The epitope specific binder can be an antibody against at least one mitochondrial membrane protein, preferably MTCO2 protein. By using an antibody against a mitochondrial membrane protein as the epitope specific binder, preferably an antibody against MTCO2, the isolated sub-population of extracellular vesicles can have increased ATP synthase activity. An isolated sub-population with an increased ATP synthase activity can be used to treat a metabolic disorder.

The epitope specific binder can be an antibody against the surface marker CD63. The isolated sub-population of CD63 positive extracellular vesicles can have reduced RNA content as compared to the extracellular vesicle bulk. An isolated sub-population with a reduced RNA content can be used to deliver cargos with minimum RNA contamination.

Sub-population of extracellular vesicles can be separated for use in therapy. In particular, the sub-population of extracellular vesicles can be separated by a method as disclosed above.

A sub-population of extracellular vesicles can be produced for use in treatment of a metabolic disorder. In particular, the sub-population of extracellular vesicles can be separated by a method as disclosed above.

### BRIEF DESCRIPTION OF THE DRAWINGS

The present invention will become more fully understood from the examples herein below and the accompanying drawings which is given by way of illustration only, and thus are not limitative of the present invention, and wherein:

FIG. 1. Schematic illustration of isolation of a sub-population of extracellular vesicles (EVs) by specific binding technique.

FIG. 2. Mitochondrial membrane proteins and canonical EV marker proteins in EV isolates detected with ELISA, indicating existence of mitochondrial protein containing sub-population of EVs.

FIG. 3. Proteomics results of sub-population of EVs. MTCO2 containing sub-population of EVs show distinct protein profile and biological process. FIG. 3A shows the number of identified proteins in the respective EV sub-populations and the number of identified proteins unique for each sub-population or present in more than one sub-population. FACL4-EV and MTCO2-EV denote sub-population of EVs isolated by FACL4 and MTCO2 antibodies, respectively. FIG. 3B shows the heat map analysis of identified proteins in the sub-populations of EVs. FIG. 3C shows the gene ontology (GO) analysis in different groups based on relative quantification of proteins.

FIG. 4. Mitochondrial proteins enriched in MTCO2 containing sub-population of EVs and the interaction of the proteins. FIG. 4A shows the relative abundance of mitochondrial proteins in different sub-populations of EVs. FIG. 4B shows the interaction of the mitochondrial proteins, with energy production machinery proteins, including subunits of ATP synthase highlighted.

FIG. 5. ATP synthase activity measurement of sub-population of EVs. MTCO2 containing sub-population of EVs has higher ATP synthase activity than non-isolated EVs.

FIG. 6. Isolation of CD63 positive EVs and the RNA profile of CD63 positive EVs. FIG. 6A shows the schematic drawing of the isolation of CD63 positive EVs and CD63 negative EVs. FIG. 6B presents RNA profile of CD63 positive and CD63 negative EVs. CD63 negative EVs contain RNAs, whereas CD63 positive EVs do not. FIG. 6C presents the relative fold change between 1st round and 4th round of CD63 and RNA signal.

FIG. 7. Sub-population of EVs contains active TGF-beta on the surface. TGF-beta was co-localized with EV markers and could induce intracellular signaling on mesenchymal stem cells. FIG. 7A shows the vesicle markers TSG101 and CD81 measured by Western Blot and the TGF-beta level measured by ELISA in corresponding fractions. FIG. 7B presents the amount of total and active form of TGF-beta in fraction 2. FIG. 7C shows the detection of the two fluorescent signals from TGF-beta and CD63. FIG. 7D presents the detection of SMAD2 phosphorylation in mesenchymal stem cells after treatment with TGF-beta containing EVs.

FIG. 8. TGF-beta containing EVs induce migration of mesenchymal stem cells (MSCs) in vitro. FIG. 8A presents microscopic images of MSC morphology change with or without the treatment of TGF-beta containing EVs. FIG. 8B presents microscopic images of MSC migration with or without the treatment of TGF-beta containing EVs. FIG. 8C shows the MSC migration results using a 48-well Boyden chamber. FIG. 8D shows the MSC invasion results using a 48-well Boyden chamber.

FIG. 9. TGF-beta on EVs is more potent than free TGF-beta for the mesenchymal stem cell migration and signaling. FIG. 9A shows numbers of migrated MSCs treated with TGF-beta containing EVs compared with the same amount of free TGF-beta. FIG. 9B shows the phosphorylation of SMAD2 in MSCs treated with TGF-beta containing EVs compared with the same amount of free TGF-beta.

FIG. 10. TGF-beta containing EVs increase migration and therapeutic potential of mesenchymal stem cells in vivo. FIG. 10A presents bioluminescence images of OVA challenged mouse model or control mice after receiving EV-treated or non-treated MSCs. FIG. 10B presents the eosinophils counts of OVA challenged mouse model or control mice after receiving EV-treated or non-treated MSCs.

FIG. 11. Motility of EVs revealed by fluorescent microscopy.

FIG. 12. Schematic illustration of generation of emptied EVs by removing intravesicular cargo.

FIG. 13. Characteristics of emptied EVs generated by removing intravesicular cargo of extracellular vesicles. FIG. 13A presents the size of EVs and emptied EVs measured by ZetaView®PMX 110. FIG. 13B presents Western Blot results of selected proteins in EVs and emptied EVs. FIG. 13C shows the RNA content in EVs as measured by Agilent Bioanalyzer. FIG. 13D shows the RNA content in emptied EVs as measured by Agilent Bioanalyzer.

FIG. 14. Electron micrograph of EV preparations treated with high pH (FIG. 14A) or revesiculated after sonication (FIG. 14B)

FIG. 15. Membrane vesicles are taken up by cultured cells through an active endocytosis process. FIG. 15A presents FACS analysis results of HEK293 cells after incubation with DiO labeled EVs. FIG. 15B presents FACS analysis results of HEK293 cells after incubation with DiO labeled membrane vesicles. FIG. 15C presents FACS analysis results of HEK293 cells after incubation with DiO labeled membrane vesicles at 37° C. FIG. 15D presents FACS analysis results of HEK293 cells after incubation with DiO labeled membrane vesicles at 4° C.

FIG. 16. Confocal microscope images of cultured cells after being incubated with fluorescently labeled EVs or fluorescently labeled membrane vesicles. Arrows indicate the green fluorescence.

FIG. 17. Loading of siRNA molecules with high pH treatment compared with PBS treatment. siRNAs are loaded into membrane vesicles more efficiently than into EVs.

FIG. 18. Membrane vesicles encompass siRNA cargo in the lumenal space. FIG. 18A presents the number of siRNAs loaded in membrane vesicle with increasing concentrations of siRNA in incubation media. FIG. 18B shows the number of siRNAs loaded in EVs and membrane vesicles. FIG. 18C shows the number of siRNAs in EVs and membrane vesicles with or without RNase A treatment.

FIG. 19. Confocal microscope images of cultured cells after incubation with fluorescently labeled cholesterol siRNA. Membrane vesicles loaded with fluorescent siRNA cargo are taken up by cultured cells. Arrows indicate the red fluorescence.

FIG. 20. RNA profiles from isolated cellular organelles show that the RNA content is similar to those of EVs.

### LIST OF ABBREVIATIONS

| Abbreviation | Meaning |
|---|---|
| PBS | Phosphate-buffered saline |
| Tris | Tris(hydroxymethyl)aminomethane |
| EDTA | Ethylenediaminetetraacetic acid |
| EV | Extracellular vesicle |
| BSA | Bovine serum albumin |
| CD9 | CD9 antigen, a cell surface glycoprotein |
| CD63 | CD63 molecule, CD63 antigen |
| CD81 | Cluster of Differentiation 81 |
| FACI4 | Fatty acid-CoA ligase 4 |
| MTCO2 | Mitochondrially encoded cytochrome c oxidase II |
| HRP | Horseradish peroxidase |
| HEPES | 4-(2-hydroxyethyl)-1-piperazineethanesulfonic acid |
| MES | 2-(N-morpholino)ethanesulfonic acid |
| M | molar, mol/liter |
| mM | millimolar, millimol/liter |
| SDS | sodium dodecyl sulfate |
| RNA | ribonucleic acid |
| DNA | deoxyribonucleic acid |
| TSG-101 | tumor susceptibility gene 101 |
| TGF-beta | transforming growth factor beta |
| SDS-PAGE | sodium dodecyl sulfate-polyacrylamide gel electrophoresis |
| TBS | tris-buffered saline |
| Tween-20 | Polyoxyethylene sorbitan monolaurate |
| AF647 | Alexa fluor ® 647 dye |
| PE | Phycoerythrin |
| HMC-1 | Human mast cell line-1 |
| Triton X-100 | polyoxyethylene octyl phenyl ether |
| SMAD2 | mothers against decapentaplegic homolog 2 |
| DAPI | 4′,6-diamidino-2-phenylindole |
| qPCR | quantitative polymerase chain reaction |
| cDNA | complementary DNA |
| EF1 | elongation factor 1 |
| LY2157299 | Gulunisertib |
| MEM | minimum essential medium |
| ECM | extracellular matrix |
| OVA | ovalbumin |
| NP40 | nonyl phenoxypolyethoxylethanol |
| 16s rRNA | 16S ribosomal RNA |
| 18s rRNA | 18S ribosomal RNA |

### EXAMPLES

### Example 1. Isolation of Sub-Population of Extracellular Vesicles by Specific Binding Technique Using Mitochondrial Membrane Proteins

### Materials and Method:

Extracellular vesicles (EVs) were isolated from Human Mast Cell line (HMC-1) by differential ultracentrifugation. Briefly, cells were grown in media containing 10% exosome-depleted fetal bovine serum for 3 days. Cell culture supernatant was centrifuged at 300×g for 10 min and 16,500×g for 20 min to remove cells and larger vesicles, respectively. The supernatant was further ultracentrifuged at 118,000×g for 3.5 hours to obtain the exosome-enriched EVs. To obtain higher purity of EVs, buoyant density gradient with OptiPrep™ (Sigma-Aldrich, St. Louis, Mo.) was conducted. EVs in PBS (1 ml) were mixed with 60% of iodixanol (3 ml) and laid at the bottom of an ultracentrifuge tube. A discontinuous iodixanol gradient (35, 30, 28, 26, 24, 22, 20%; 1 ml each, but 2 ml for 22%) in 0.25 M sucrose, 10 mM Tris, and 1 mM EDTA was overlaid, and finally the tubes were filled to completion with approximately 400 μl of PBS. Samples were ultracentrifuged at 178,000×g for 16 hours. Mixture of fractions 2 and 3 (from top) were diluted with PBS (up to 94 ml) and ultracentrifuged at 118,000×g for 3.5 hours. The pelleted EVs were resuspended in PBS.

First, existence of mitochondrial membrane proteins in the EV isolates was confirmed by ELISA. EVs were coated on 96 well plates overnight at 4° C. Then the plate was blocked with 1% BSA in PBS for 1 hour at room temperature and further incubated with antibodies against CD9, CD81, FACL4, or MTCO2 for 2 hours at room temperature. After washing with PBS, HRP conjugated secondary antibodies were incubated for 1 hour at room temperature. Plates were washed with PBS and then developed by colorimetric reaction of HRP.

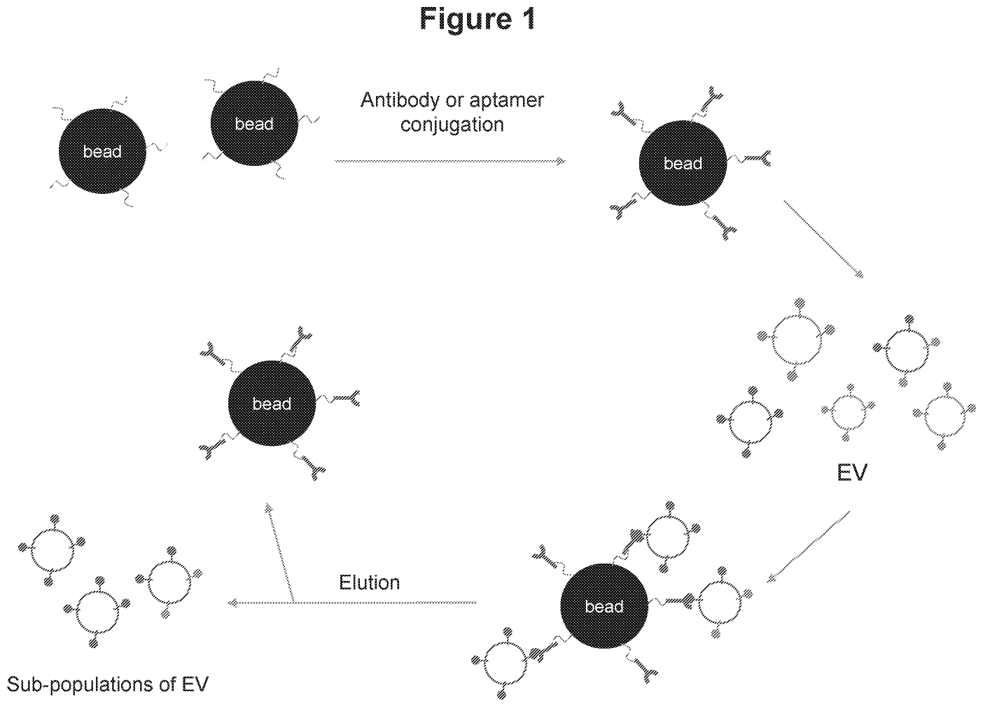

To isolate the mitochondrial proteins containing sub-populations of EVs, specific antibodies against mitochondrial membrane proteins were used as shown in FIG. 1. Antibodies of FACL4 and MTCO2 were coupled to Dynabeads according to the manufacturer's instructions (Thermo Fischer). Antibody coupled beads were incubated with EVs for 2 hours at room temperature. Unbound EVs were removed and washed with PBS twice. Bead bound EVs were eluted with acidic washing buffer (10 mM HEPES, 10 mM MES, 120 mM NaCl, 0.5 mM MgCl2, 0.9 mM CaCl2, pH5).

### Results:

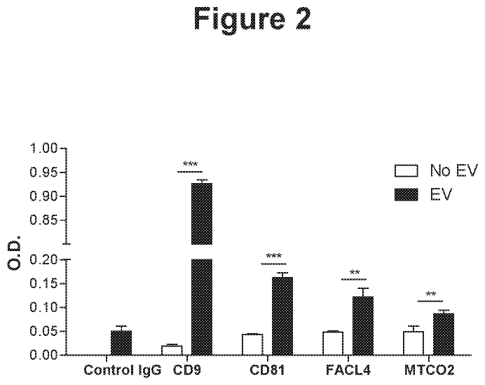

The markers of EVs. CD9 and CD81, were detected with ELISA, which is indicated by optical density (O.D.). In addition, mitochondrial membrane proteins, FACL4 and MTCO2, were detected (FIG. 2). This result shows that mitochondrial membrane proteins are present in the EV isolates and can be detected and isolated with those antibodies.

### Example 2. Proteomic Analysis of Sub-Population of Extracellular Vesicles

### Materials and Methods:

The proteome of isolated mitochondria protein containing sub-populations of EVs were identified by LC-MS/MS. Briefly, 10 μg of vesicles of non-isolated (EV), FACL4-isolate (FACL4-EV), and MTCO2-isolated (MTCO2-EV) EVs were lysed with 2% SDS and sonicated. Tryptic digestion of proteins was conducted by Filter Aided Sample Preparation. Digested peptides were analyzed with an OrbiTrap mass spectrometer. Peak lists of MS data were generated and peptides/proteins were identified and quantified using the MaxQuant quantification tool with Andromeda search engine (version 1.5.2.8). The search parameters used were as follows: enzyme specificity, trypsin; variable modification, oxidation of methionine (15.995 Da) and the carbamidomethylation of cysteine (57.021 Da); two missed cleavages; 20 ppm for precursor ions tolerance and 4.5 ppm for fragment ions tolerance. Homo sapiens reference proteome set from Swiss-Prot database (20196 entries), contaminants, and reverse sequences were used for search. For peptide and protein identification, 1% false discovery rate was determined by accumulating 1% of reverse database hits. To obtain the quantitative data, label-free quantification (LFQ) with a minimum of two ratio counts was applied. Normalized LFQ intensity was obtained. Biological process terms of gene ontology (GO) analysis was obtained using DAVID available at the david.ncifcrf.gov website (https://david.ncifcrf.gov/).

### Results:

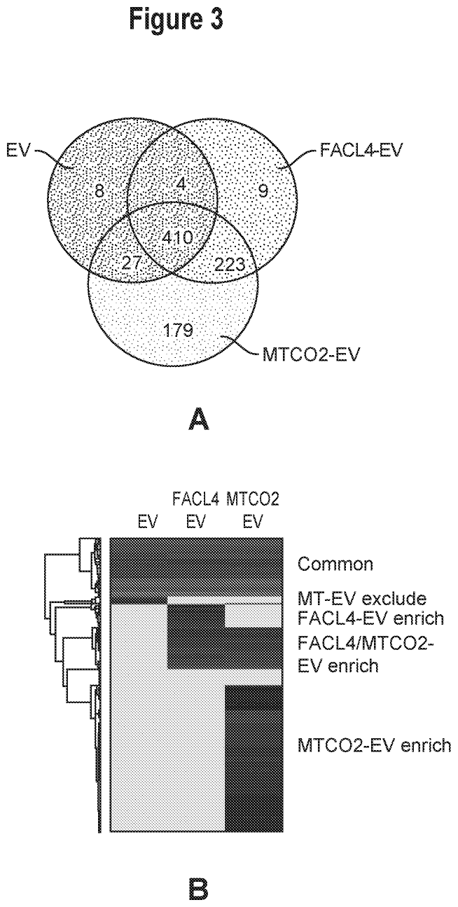

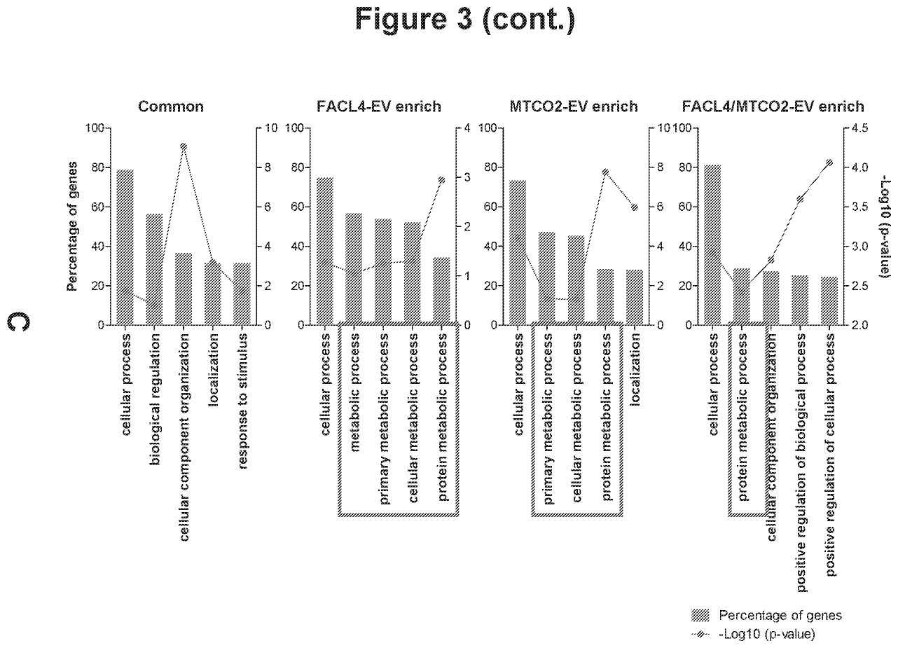

In total, 449, 646, 839 proteins were identified from EV, FACL4-EV, and MTCO2-EV, respectively. Overlapping proteins between samples is presented in FIG. 3A. In addition, heatmap analysis of vesicles is shown in FIG. 3B. Based on relative quantification of proteins, proteins were categorized in 5 different groups. Among them, ‘common’, ‘FACL4-EV enrich’, ‘MTCO2-EV enrich’, and ‘FACL4/MTCO2-EV enrich’ were further analyzed with gene ontology (FIG. 3C). Most of proteins were common in all 3 vesicles and EV and FACL4-EV were very similar. However, MTCO2-EV was different from the other 2 types of vesicles. Importantly, mitochondrial protein containing EVs were enriched with metabolic process related proteins, compared with non-isolated EVs. These results indicate that sub-populations of EVs have different protein cargos and can thus mediate different biological functions.

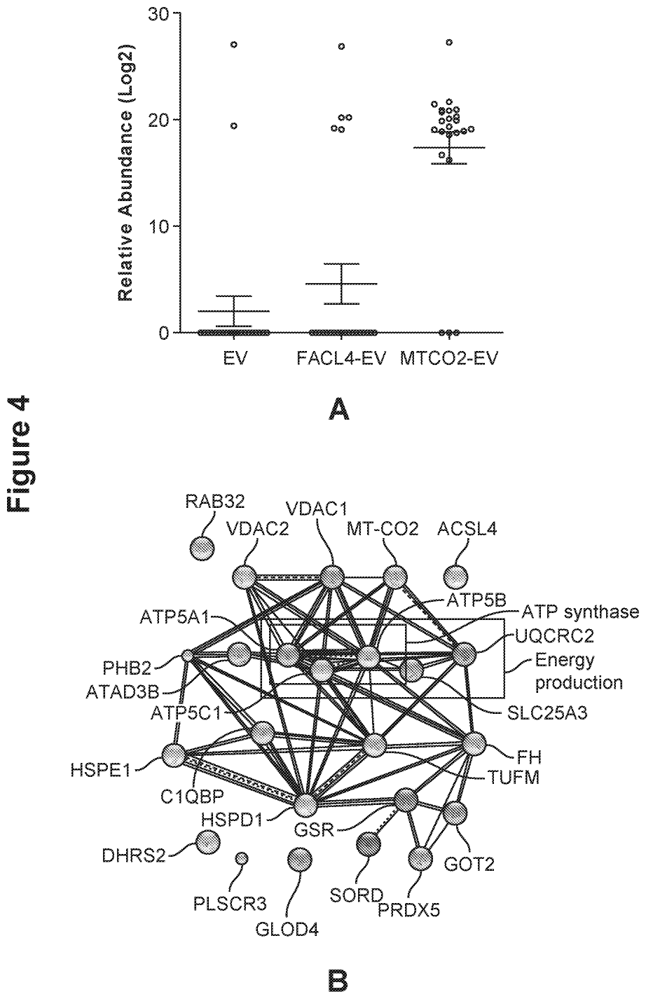

Mitochondrial proteins were identified with higher abundance in MTCO2-EV compared with the other 2 types of vesicles (FIG. 4A) and were physically bound to each other (FIG. 4B). Importantly, energy production machinery proteins including subunits of ATP synthase were found in MTCO2-EV (FIG. 4B).

### Example 3. ATP Synthase Activity of Sub-Population of Extracellular Vesicles

### Materials and Methods:

One of the MTCO2-EV enriched mitochondrial proteins which were found from LC-MS/MS was ATP synthase. The activity of ATP synthase was tested with ATP synthase enzyme activity microplate assay kit (Abcam) according to the manufacturer's instructions. Non-isolated EV, MTCO2-EV, and MTCO2-unbound EV were subjected to the test and the relative activity was measured.

### Results:

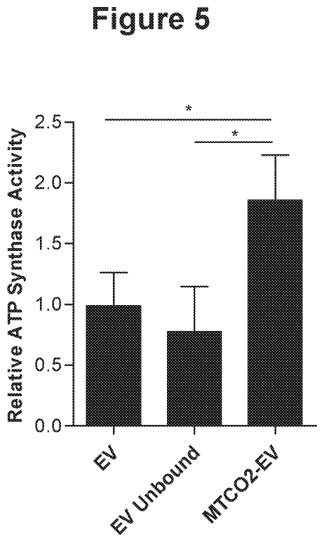

Compared with non-isolated EV, MTCO2-EV has around 2 fold higher ATP synthase activity (FIG. 5). In addition, MTCO2-unbound EV has slightly reduced ATP synthase activity, although this is not statistically significant. This result suggests that mitochondrial protein containing sub-population of EVs are enriched with active ATP synthase.

### Example 4. Isolation and RNA Profiling of CD63-Positive Extracellular Vesicles

### Materials and Methods:

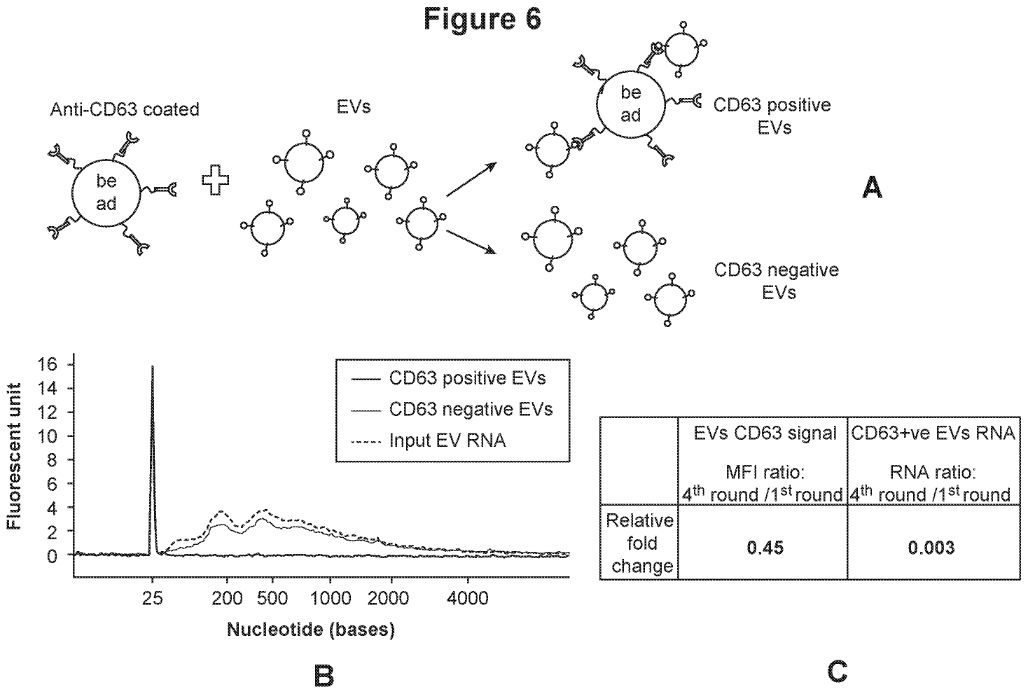

CD63 is a classical marker for EVs. We used anti-CD63 coated magnetic beads to capture a CD63 positive EV subset. 100 μg of EVs were incubated with 107 magnetic beads (Life-technology, 10606D) overnight at 4 degree with gentle rotation (FIG. 6A). Beads were removed and fresh beads were added. These steps were repeated for 3 more times. Later, CD63 positive EVs on beads and CD63 negative supernatant were subjected to RNA isolation using Exiqon total plant and animal cells kit as per manufacture recommendations. RNA size profiles were compared using bioanalyzer with nano-chip. Gain of EVs associated proteins or EVs associated RNA signal bounded on CD63 beads was evaluated by measuring the gain in CD63 fluorescent signal in EVs bounded beads and RNA measurements from round the first and the fourth round of capture.

### Results:

Our finding indicated that bioanalyser RNA profile showed no/un-detectable traces of RNA in CD63 positive EVs (FIG. 6B). This suggests presence of CD63 positive EVs subsets do not contain any detectable RNA. Whereas the CD63 negative EVs had majority of RNA nucleotides present in bead unbounded form.

To further confirm the capture of CD63 positive EVs on beads we measured the mean fluorescence signal of CD63 using flow cytometry and RNA from first round of capture and 4th round of capture. From the ratio between 1st round and 4th round of CD63 and RNA signal showed ˜50% and ˜0.3% increase respectively (FIG. 6C). Taken together this data indicate that CD63 positive EVs devoid of RNA that are usually considered to be the part of EVs.

### Example 5. Sub-Population of Extracellular Vesicles Contain TGF-Beta

### Materials, Methods and Results:

EVs were isolated from HMC-1 by differential ultracentrifugation as described in example 1. After OptiPrep™ gradient, each fraction was obtained. Vesicle markers (TSG101 and CD81) and TGF-beta level were measured by Western Blot and ELISA, respectively. For the Western Blot, each fraction of OptiPrep™ gradient were subjected to SDS-PAGE and transferred onto Nitrocellulose membranes. Membranes were blocked with 5% BSA in TBS containing 0.05% Tween-20 and incubated with primary antibodies for overnight at 4 degree. After washing with TBS containing 0.05% Tween-20, HRP conjugated secondary antibodies for 1 hour at room temperature. Immunoreactive bands were visualized. Levels of TGF-beta 1 (total and active form) in vesicles were performed using a TGF beta 1 ELISA Ready-SET-Go kit (eBioscience, Affymetrix, Inc) according to the instruction of the manufacturer.

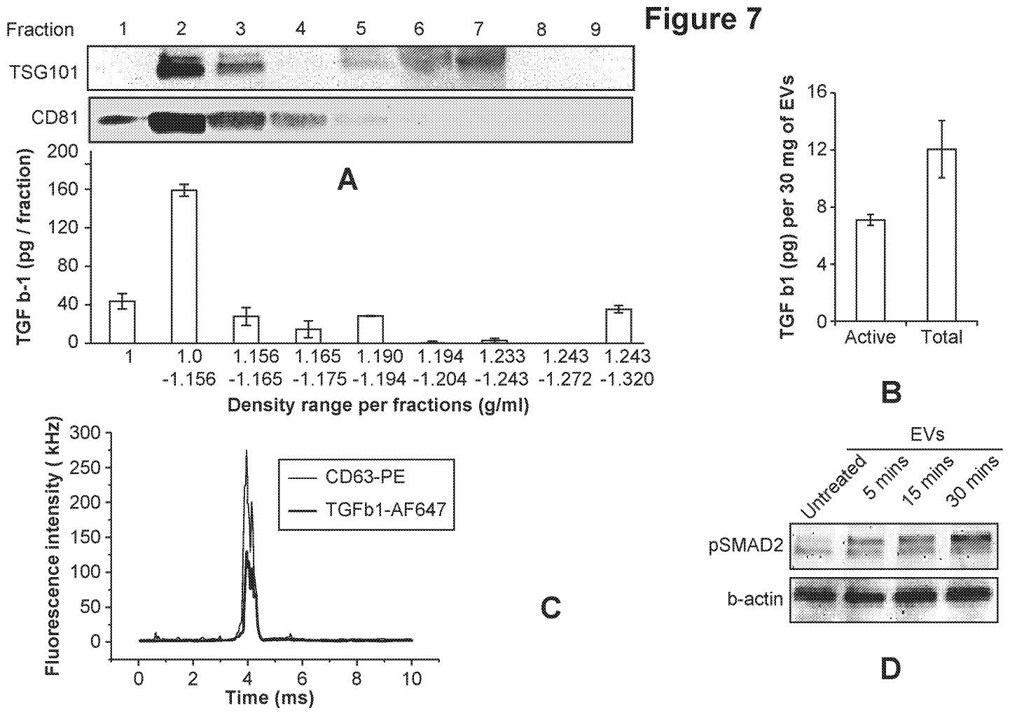

Both TSG101 and CD81 were found in fraction 2 mostly but also in other fractions (FIG. 7A). In addition, most of TGF-beta was found in fraction 2 with active form of TGF-beta (FIGS. 7A and 7B). These results show that EVs harbor active TGF-beta.

Colocalization of EVs and TGF-beta, fluorescent correlation spectrometry was conducted. Freshly isolated EVs were labeled with TGFbeta-AF647 and CD63-PE. The labeled EVs were loaded from bottom on OptiPrep™ cushion (0, 20, 30, 50%) and centrifuged at 40.000 rpm for 4 hours (SW40-Ti Rotor) to separate them from free unbound dye. The lipid labeled vesicles were collected from 20-30% and washed in PBS for 120,000×g for 3.5 hours. Washed pellet was subjected to custom designed two color fluorescent correlations spectroscopy system with configuration microscope system. As shown in FIG. 7C, two fluorescent signals from TGF-beta and CD63 were detected at the same time points, implicating that EVs harbor TGF-beta on their surface.

Next, activity of TGF-beta on vesicles was examined by treating them to mesenchymal stem cells (MSCs). MSCs were grown to 70-80% confluence. After washing with PBS, EVs from HMC-1 cells (100 μg/ml) were treated. At 0, 5, 15, and 30 minutes after treatment, downstream signal of TGF-beta was analyzed by Western Blot. One of important TGF-beta downstream signal molecules, SMAD2, was phosphorylated with time-dependent manner (FIG. 7D).

In summary, EVs harbor active TGF-beta on their surface. TGF-beta can induce the intercellular signaling via TGF-beta type-1 receptor and SMAD2.

### Example 6. TGF-Beta Containing Sub-Population of Extracellular Vesicles Induce Migration of MSCs In Vitro with Higher Activity than Free TGF-Beta

### Materials, Methods and Results:

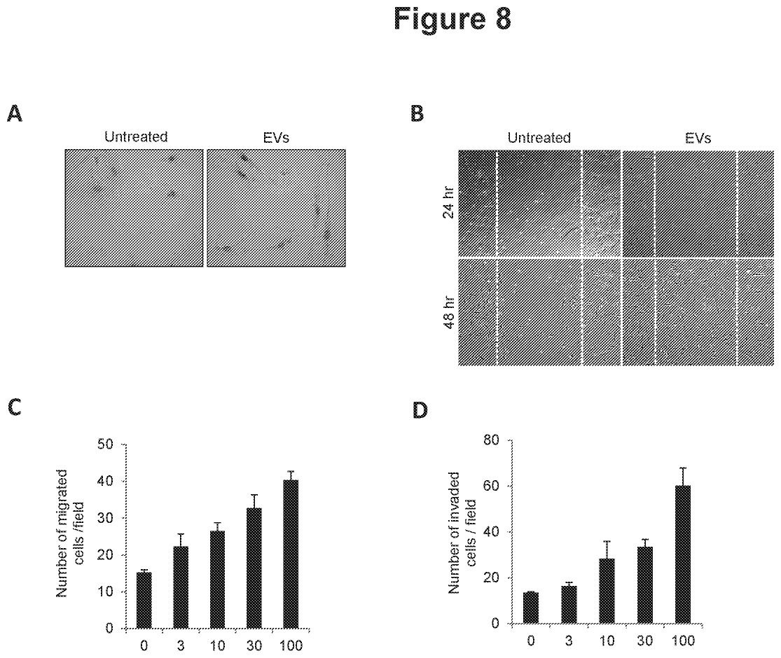

MSCs were treated with EVs and their morphology change was observed with microscopy (FIG. 8A). Cells were more elongated after treatment. MSCs were grown to 70-80% confluence in 6 well plates and the monolayer cells were scratched with a 1 ml pipette tip across the center of wells. After washed with PBS, MEM plain medium with or without EVs from HMC-1 cells (100 tag/ml) was added to plates. Migratory cells from the scratched boundary were imaged after 24 and 48 hours. EV-treated MSCs showed increased wound healing activity compared with non-treated MSCs (FIG. 8B).

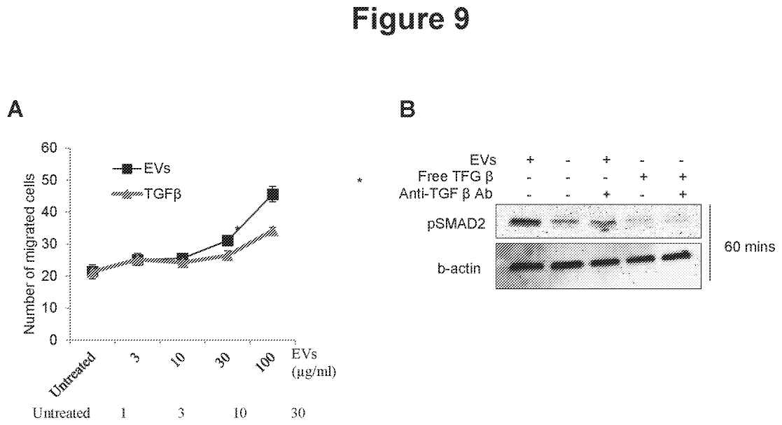

MSCs migration and invasion were evaluated using a 48-well Boyden chamber (Neuroprobe Inc). Cells (5000 cells/well) were seeded to the bottom compartment and was separated from the upper part by a polycarbonate membrane with 8 μm pores. The membrane was pre-coated with 0.1% gelatin or 200 μg/ml ECM Gel from Engelbreth-Holm-Swarm murine sarcoma (Sigma-Aldrich). After seeding, cells were allowed to adhere onto the membrane by inverting the chamber assembly upside down for 3.5 hours. Later the chamber was placed in correct orientation and EVs were added in the upper compartment. After incubation for 12 hours at 37° C., the membrane was removed and cells on the migrated sides were fixed in methanol (10 mins), and stained with Giemsa (Histolab) for 1 hour. Cell from the non-migrated side were wiped out before imaging. Three fields at 40× magnification were imaged. Migration and invasion of MSCs were significantly increased by EV treatment in dose-dependent manner (FIGS. 8C and 8D). This migratory activity was higher if MSCs were treated with EVs, compared with same amount of free TGF-beta (FIG. 9A). Furthermore, phosphorylation of SMAD2 was prolonged in EV-treated MSCs (FIG. 9B).

Collectively, TGF-beta containing sub-population of EVs induce the MSC migratory activity in vitro and this activity is more potent if TGF-beta is localized in the EVs.

### Example 7. TGF-Beta Containing Sub-Population of Extracellular Vesicles Increase Migration and Therapeutic Efficacy of MSCs In Vivo

### Materials and Methods:

OVA challenged mouse model of lung inflammation was used to evaluate the migration and therapeutic potential of EV-treated MSCs. Intra-peritoneal (i.p) injection OVA (8 μg/body) were performed to sensitized mouse on day 1. On three consecutive days (14, 15 and 16 day) the mouse was intra-nasally (i.n) exposed to 100 μg/body OVA (OVA/OVA group) or with PBS (OVA/PBS). On Day 17 post sensitization mouse from each group received 0.5 Million MSCs (expressing constitutive Luciferase and Green Fluorescent Protein) that are either incubated or not incubated with EVs for 48 hours. After 10 mins, 30 mins and 60 mins, bioluminescence (photons/sec/cm2) from whole body of the mice was acquired with IVIS spectrum (Caliper Life Sciences). Three days later, mice were sacrificed and eosinophils in Bronchoalveolar lavage (BAL) fluid were counted.

### Results:

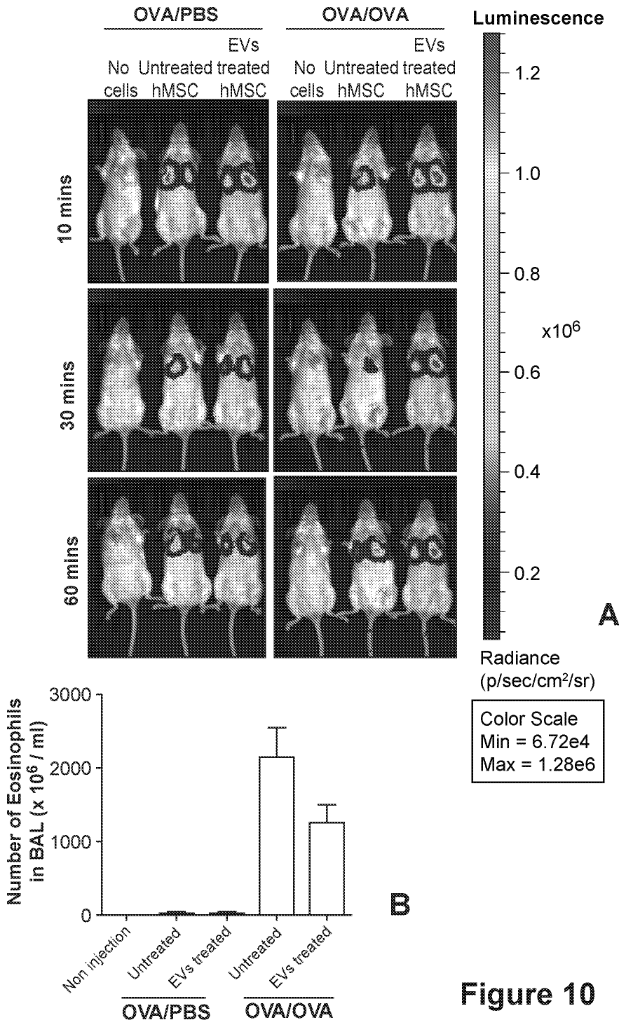

Migration of MSCs to the inflamed lung tissue was higher in EV-treated MSCs compare with non-treated MSCs in OVA/OVA group at 10, 30, and 60 minutes (FIG. 10A). However, there was no difference between EV-treated and non-treated MSCs in OVA/PBS group. After 3 days of injection, therapeutic activity of MSCs was evaluated by eosinophils counting in BAL fluid. As compared with non-treated MSCs injected mice, EV-treated MSCs injected mice showed lower eosinophils numbers in OVA/OVA group (FIG. 10B). These results suggest that TGF-beta containing EVs increase the migratory activity of MSCs toward inflamed tissue, thereby enhance the therapeutic efficacy of MSCs.

### Example 8. Motility of Sub-Population of Extracellular Vesicles

### Materials and Methods:

EV was pelleted down at 16,500×g, re-suspended and further diluted in PBS. A volume of 100 μl was then placed in the center of a glass bottom culture dish (35 mm petri dish, 14 mm microwell, no. 1.0 coverglass (1.13-1.16 mm), MatTek Corporation) and left to sediment for 15 minutes at room temperature. The glass bottom dish was then gently washed three times with PBS. PKH67 dye diluted in Diluent C (Sigma-Aldrich) 1:1000 was added to the center of the glass bottom dish in a volume of 500 μl and left to incubate for 5 minutes at room temperature. The dishes were then again gently washed three times with PBS after which the sample was immediately evaluated under the microscope (Axio Observer.Z1, Zeiss). Time-lapse photos were acquired with 30 second intervals over a period of 8 minutes to monitor the motility of vesicles in the sample.

### Results:

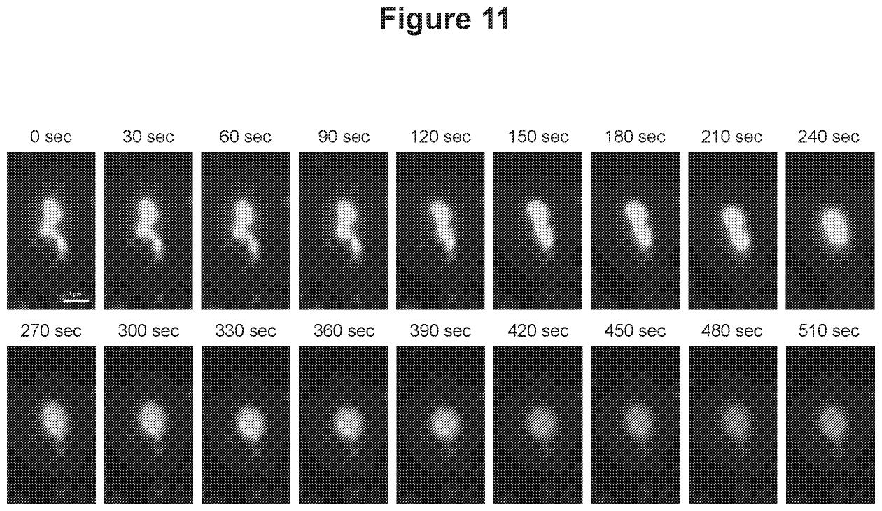

EVs that were labeled with PKH67 dye were visualized with green fluorescent signal and changed their morphology over a period of times (FIG. 11). This result indicates that a sub-population of EVs has motile activity.

### Example 9. Generation of Emptied EV by Removing Intravesicular Cargo of Extracellular Vesicles

### Materials and Methods:

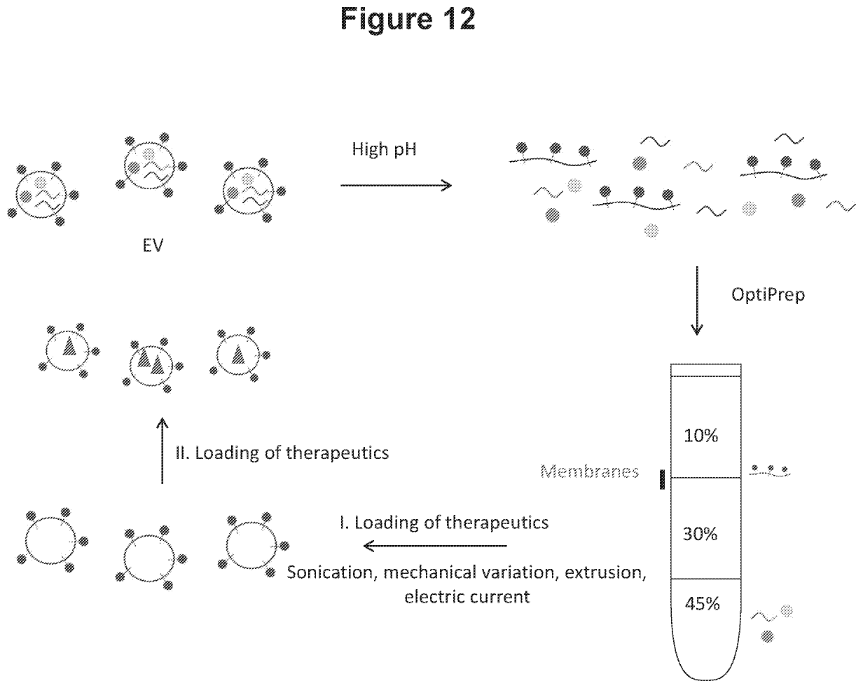

Schematic illustration of generation of therapeutic membrane vesicles is shown in FIG. 12. EVs from HMC-1 cells were incubated with high pH solution (200 mM sodium carbonate, at pH 11) for 2 hours at room temperature. To collect the membrane only, OptiPrep™ density gradient was conducted. Sample was mixed with 60% OptiPrep™ to make 45% OptiPrep™. Mixed 45% OptiPrep™ was laid on the bottom and overlaid with 10 and 30% OptiPrep™. Sample was ultracentrifuged at 100,000×g for 2 hours. Membranes were obtained from interface of 10 and 30% OptiPrep. Isolated membranes were re-vesiculated by sonication.

### Results:

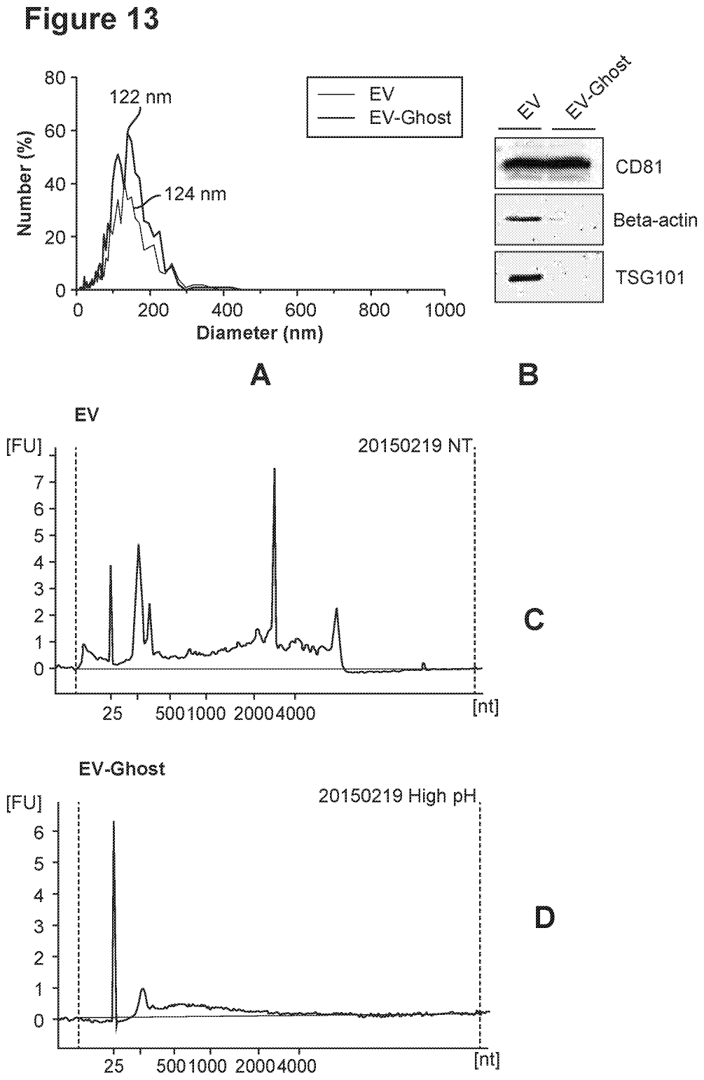

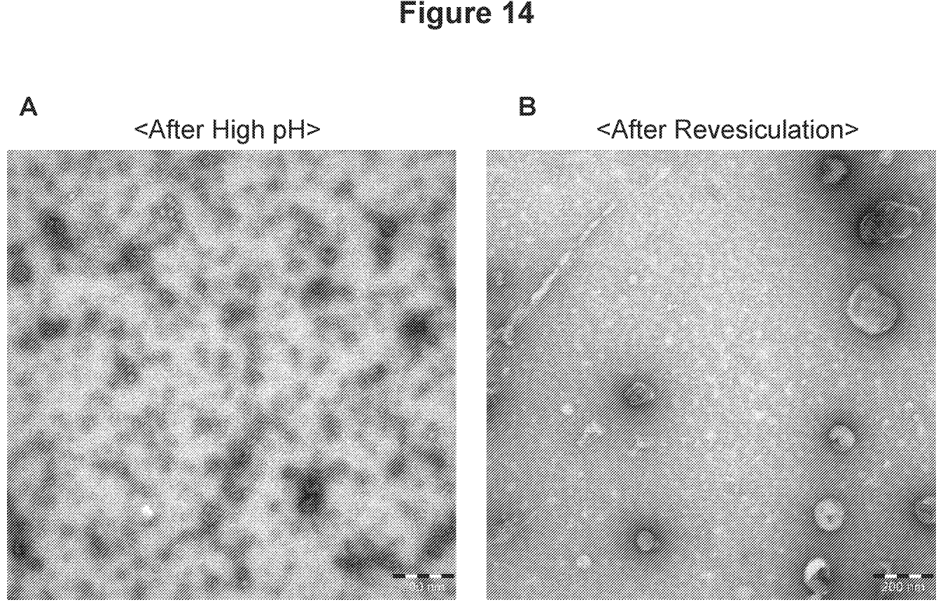

The size of EVs and emptied EV which was measured using ZetaView® PMX 110 (Particle Matrix) was similar and showed median size at 124 and 122 nm, respectively (FIG. 13A). Intravesicular cargo, proteins and RNA, were analyzed by Western Blot and Bioanalyzer, respectively. Emptied EVs had reduced intravesicular proteins, beta-actin and TSG101, but still contained the membrane protein CD81 (FIG. 13B). Furthermore, EVs contained abundant RNA (FIG. 13C), but emptied EVs contained almost no RNA as measured by Agilent Bioanalyzer (FIG. 13D). Additionally, electron microscopy confirmed that EV membranes collected after high pH treatment did not form vesicles (FIG. 14A), while EVs that were processed and re-vesiculated by sonication readily formed vesicles with typical exosomes characteristics (FIG. 14B). From these results, we could conclude that membrane of EVs without intravesicular cargo can be obtained by high pH treatment and can be reassembled.

### Example 10. Cellular Uptake of Re-Vesiculated Membrane Vesicles

### Materials and Methods:

EVs from HEK293T cells were incubated with high pH solution (200 mM sodium carbonate (aq.), at pH 11) for 2 hours at room temperature. To label the membrane, lipophilic dye. DiO (5 μM), was added and incubated for 1 hour at room temperature. The sample was subsequently mixed with 60% (w/V) iodixanol to obtain a sample solution containing 45% (w/V) iodixanol. The sample solution was placed at the bottom of a centrifuge tube and a 10% (w/V) iodixanol solution followed by a 30% (w/V) iodixanol solution were added on top of the sample solution to form a density gradient. The tube with its contents was subsequently ultracentrifuged at 100,000×g for 2 hours to obtain membranes from the interface between the 10% (w/V) and the 30% (w/V) iodixanol layer. The isolated membranes were subjected to sonication to reassemble membrane vesicles. At the same time, EVs from HEK293T cells were incubated with DiO (5 μM) and purified by an iodixanol density gradient as described above but without high pH treatment. The number of membrane vesicles and EVs was measured by ZetaView® instruments.

For FACS analysis, HEK293T cells (1×10⁵ cells) were seeded on 24 well plates and incubated overnight. Different number of DiO labeled membrane vesicles or EVs were incubated with the cells for 1 hour at 37 or 4° C. Cells were washed with PBS once, trypsinized, and then fixed by 4% paraformaldehyde for 10 min at room temperature. DiO signal in the cells was analyzed by FACS.

For confocal microscopy, HEK293T cells (1×10⁵ cells) were seeded on glass cover slips on 24 well plates and incubated overnight. DiO labeled membrane vesicles (1×10⁸/ml) or EVs (1×10⁸/ml) were incubated for different time points (3, 6, 12, 24 hours) at 37° C. Cells were stained with CellMask Deep Red Plasma membrane staining dye for 10 min at 37° C. Cells were washed with PBS once and fixed by 4% paraformaldehyde for 10 min at room temperature. Glass cover slips were mounted on slides with ProLong® Diamond Antifade Mountant with DAPI. Fluorescence was observed by confocal microscopy.

### Results:

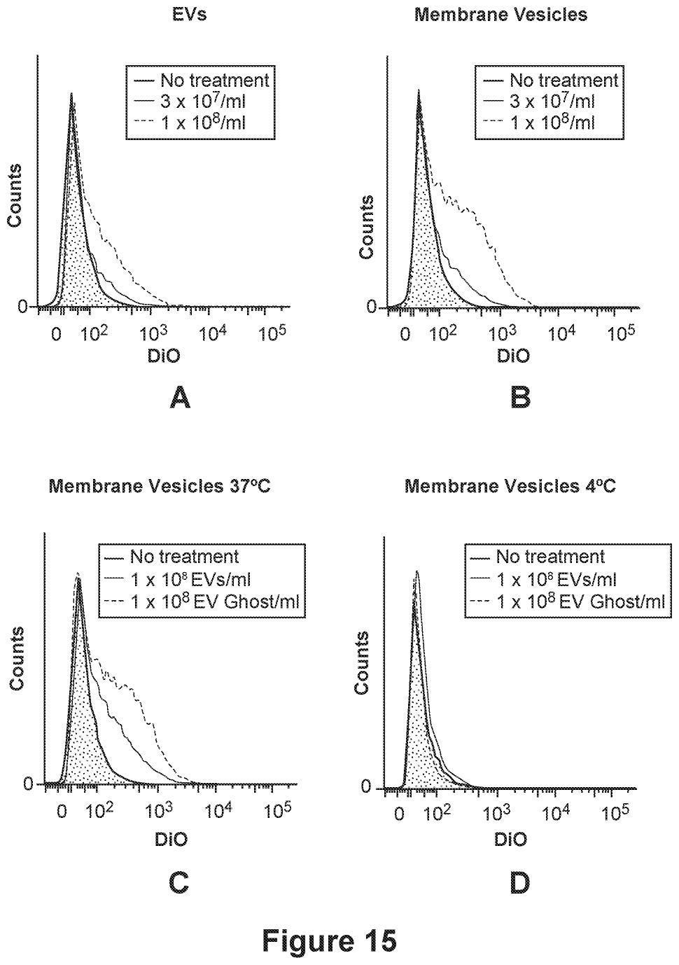

FACS data showed that both EVs and membrane vesicles were efficiently taken up by the recipient HEK293T cells after a 1 hour incubation. Membrane vesicles showed higher fluorescent signal compared to EVs (FIGS. 15A-B). This uptake was totally abolished by 4° C. incubation, which suggests that uptake is involved with active endocytosis mechanism (FIGS. 15C-D).

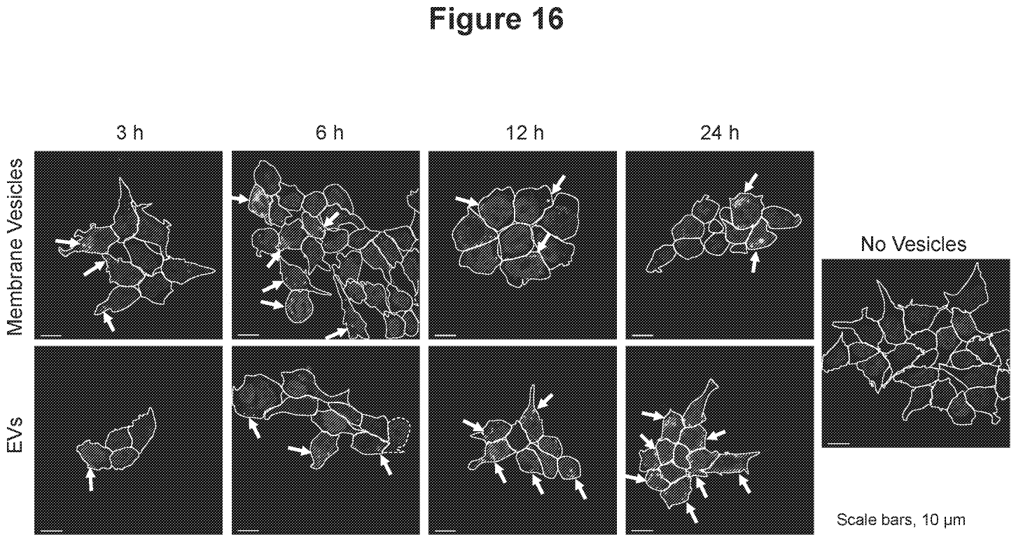

Confocal data showed that both membrane vesicles and EVs were taken up by HEK293T cells, but uptake of membrane vesicles was faster than EVs (FIG. 16).

### Example 11. Loading Cholesterol-siRNA into Membrane Vesicles

### Materials and Methods:

EVs from HEK293 cells were diluted to 1×10¹²/ml and incubated in either PBS or 0.1M sodium bicarbonate pH 11 for two hours at room temperature. The preparations were pelleted at 100,000×g for 15 min at 4° C. and the resulting pellet was washed once and resuspended in PBS. The two preparations were incubated with increasing amounts of Alexa 647-labeled siRNA targeting luciferase, ranging from 0.5 μM to 5 μM. For the EVs suspended in PBS, the preparations were mixed at 37° C. for 1 hour at 450 RPM. Each sample was then spun at 100,000×g for 15 minutes to pellet the EVs, the supernatant was removed, and the pellet was resuspended in PBS. For the EVs treated at ph11, the preparations were sonicated for 30 minutes and purified on an iodixanol gradient as described in Example 10, above. All samples were resuspended in PBS and aliquoted in a 96-well plate, which was analyzed for total fluorescence signal (excitation at 647 nm, emission at 675 nm) and plotted against an Alexa 647 standard curve.

### Results:

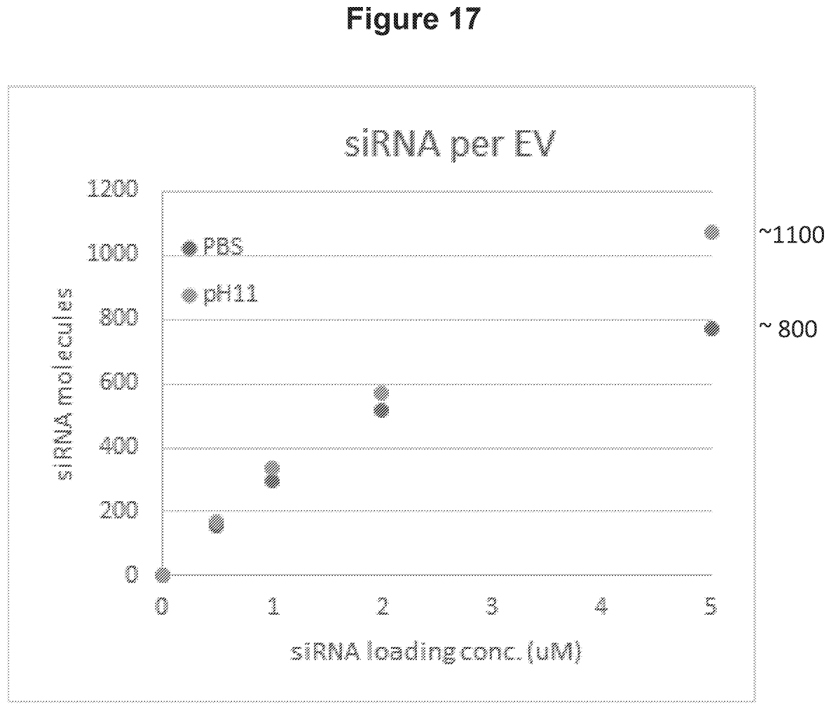

As shown in FIG. 17, both the EVs resuspended in PBS and the EVs treated at pH11 bind the fluorescent siRNA in a dose-dependent manner. At all concentrations measured, the EVs treated at pH 11 had a higher fluorescent signal than the EVs in PBS. At the highest siRNA concentration used (5 μM), the pH11 vesicles contained about 1,100 siRNA molecules per vesicle compared to about 800 siRNA molecules per vesicle for the PBS vesicles. These results indicate that treating EVs with high pH allows for higher loading efficiency than unmodified EVs.

### Example 12. Lumenal Protection of siRNA in High pH Treated Membrane Vesicles

### Materials and Methods:

EVs from HEK293T cells were incubated with high pH solution (200 mM sodium carbonate (aq.), at pH 11) for 2 hours at room temperature. Different concentration (0, 0.6, 2, 6, 20, 60 μM) of Cy3-labeled cholesterol-siRNAs against cMyc were added and incubated for 1 hour at 37° C. Membranes were isolated by iodixanol density gradient as described above. The isolated membranes were subjected to sonication to reassemble membrane vesicles. The number of membrane vesicles was measured by ZetaView® instruments. The number of siRNAs on membrane vesicles was calculated using by fitting to a fluorescence intensity standard curve, which was measured by Varioscan instrument at excitation/emission of 650 nm/670 nm.

EVs from HEK293T cells were incubated with Cy3-labeled cholesterol-siRNAs (60 μM) for 1 hour at 37° C. and purified by an iodixanol gradient as described above. The number of siRNAs was calculated with same method as described above.

Membrane vesicles and EVs loaded with Cy3-labeled cholesterol-siRNAs (60 μM) were incubated with RNase A (10 μg/ml) for 20 min at 37° C. and then further isolated by iodixanol density gradient. The remaining fluorescent intensity was measured and number of siRNAs was calculated.

### Results:

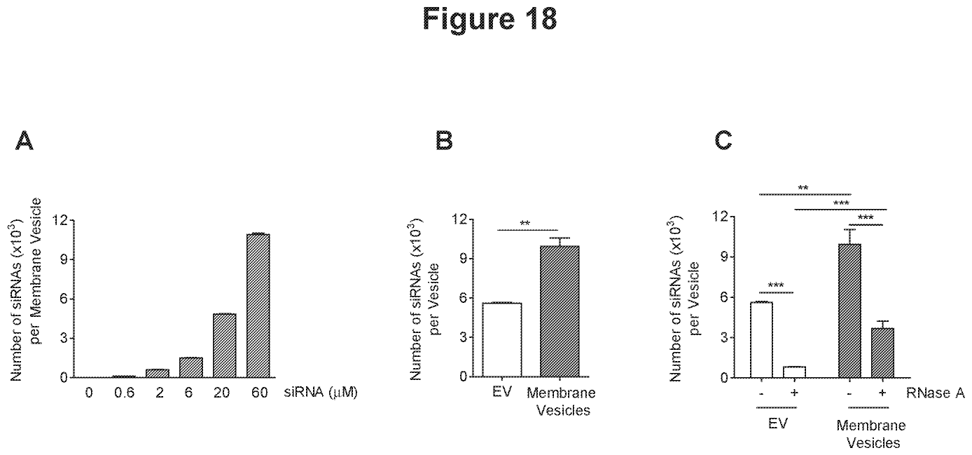

Loading of membrane vesicles was highly dependent on the siRNA concentration. As shown in FIG. 18A, the average number of siRNA molecules per membrane vesicle reached as high as ˜10,000 when the membrane vesicles were used at the highest concentration of 60 μM. At this concentration, membrane vesicles were loaded with siRNA more efficiently than EVs (FIG. 18B). Importantly, a significant amount of the siRNA signal associated with EVs was removed after RNase A treatment, while the siRNA signal from membrane vesicles was more moderately reduced after RNase A treatment (FIG. 18C). These results suggest that the membrane vesicles incorporated the siRNA both onto the vesicle surface as well as within the lumen of the vesicle, while EVs only incorporated the siRNA onto their outer surface. This method demonstrates that membrane vesicles can be more efficiently loaded with macromolecular cargo than EVs.

### Example 13. Uptake of Cholesterol-siRNA Loaded Membrane Vesicles

### Materials and Method:

Membrane vesicles were loaded with Cy3-labeled cholesterol-siRNAs (60 μM) as described in Example 12, above. HEK293T cells (1×10⁵ cells) were seeded on glass cover slips on 24 well plates and incubated overnight. Membrane vesicles loaded with the siRNA (5×10⁸/ml) were incubated for different durations (3, 6, 12, 24 hours) at 37° C. Cells were washed with PBS once and fixed by 4% paraformaldehyde for 10 min at room temperature. Glass cover slips were mounted on slides with ProLong® Diamond Antifade Mountant with DAPI. Fluorescence was observed on confocal microscopy.

### Results:

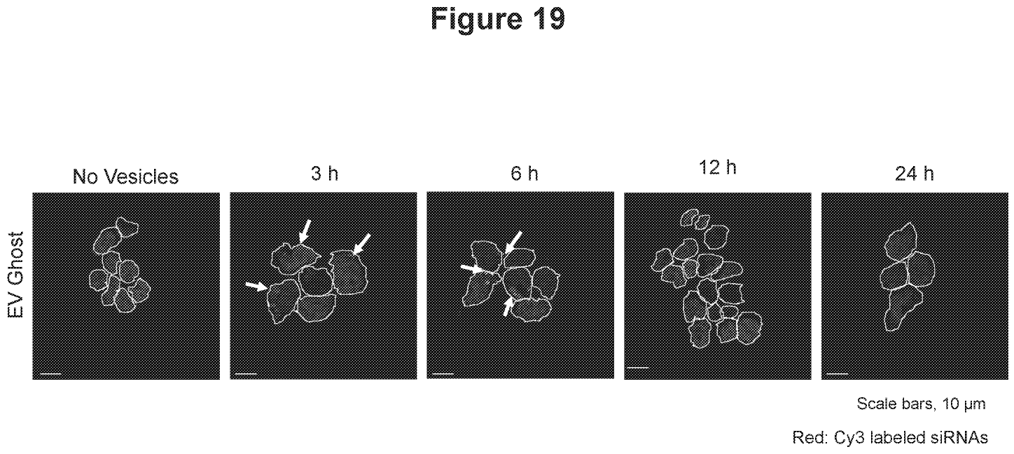

Fluorescent signal in the cytoplasm of the recipient cells was observed most intensely at 3 and 6 hours after treatment, but was decreased at 12 and 24 hours (FIG. 19). The kinetics of fluorescent siRNA uptake signal was similar to the uptake of membrane vesicles as described in Example 10, above. This result indicates that membrane vesicles loaded with siRNAs were efficiently delivered to the cytoplasm of recipient cells.

### Example 14. Isolation of Organelles from Cells

### Materials and Methods:

Crude organelle preparations were made from HMC-1 cells. Briefly, cells were washed with PBS and suspended in ice cold buffer-I (150 mM NaCl, 50 mM HEPES pH 7.4 and 25 μg/ml Digitonin) for 20 minutes in ice and then centrifuged at 2,000×g to pellet the cells. This pellet was incubated with Buffer-II (150 mM NaCl, 50 mM HEPES pH 7.4 and 1% NP40) for 40 minutes in ice and centrifuged at 7,000×g to pellet nuclei and cellular debris. The supernatant containing crude membrane-bound organelles was enriched in Endoplasmic reticulum (ER), Golgi, Mitochondria and some nuclear luminal proteins. This fraction was mixed with 60% iodixanol and loaded below various percentages of OptiPrep™ to form a density gradient (0, 20, 22, 24, 26, 28, 30, 35 and 50%) and ultracentrifuged for 16 hours at 178,000×g. Ten fractions (top to bottom) were collected and subjected to RNA isolation using Exiqon total plant and animal cells kit (Exiqon). Distribution of RNA traces across various floating densities was determined by Bioanalyzer profile.

### Results:

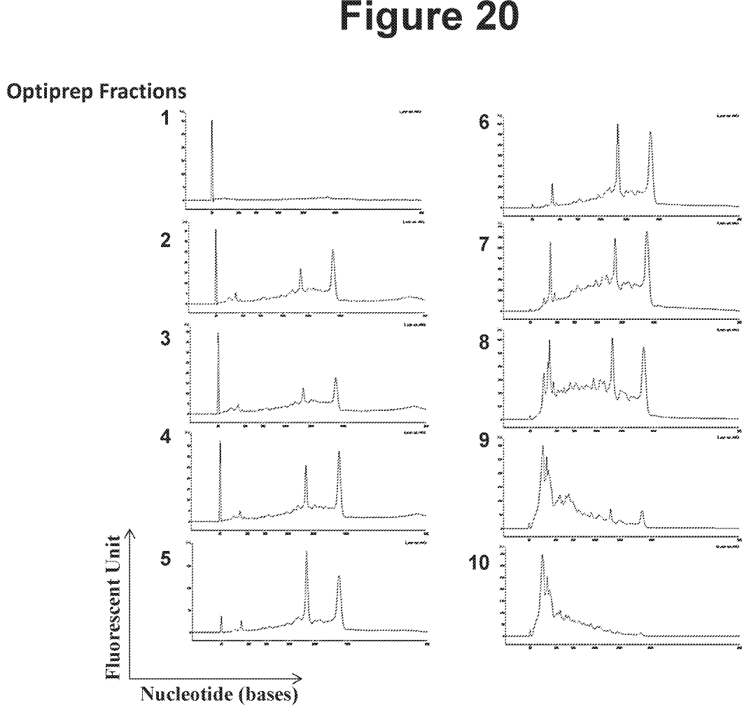

As shown in FIG. 20, the distribution of RNA in the gradient was quite broad and RNA traces were found across the gradient. Interestingly, long RNA (16s and 18s rRNA) sequences were enriched in low density fractions but short RNA stretches were highly enriched in high density fractions. The overall distribution of crude organelles was similar to the RNA profiles seen from EV preparations. This RNA-based distribution data shows that cellular organelles contribute to a subset of EVs in a mixed EV population.

1. A method for producing membrane vesicles comprising:
a. providing extracellular vesicles or organelles;
b. opening said extracellular vesicles or said organelles by treatment with an aqueous solution having a pH ranging from 9 to 14 to obtain membranes;
c. removing intravesicular or organellar content; and
d. re-assembling said membranes to form membrane vesicles.

2. The method according to claim 1, wherein step d is done by one or more of sonication, mechanical vibration, extrusion through porous membranes, electric current and combinations thereof.

3. The method according to claim 2, further comprising
e. loading a cargo into said membrane vesicles, wherein step e can be performed concomitantly with or after step d.

4. The method according to claim 3, wherein step e is done by physical manipulation after step d, wherein said physical manipulation is selected from electroporation, sonication, mechanical vibration, extrusion through porous membranes, application of electric current and combinations thereof.

5. The method according to claim 3, wherein said cargo is selected from a synthetic bioactive compound, a natural bioactive compound, an antibacterial compound, an antiviral compound, a protein, a nucleotide, a genome editing system, microRNA, siRNA, long-non-coding RNA, antago-miRs, morpholino, mRNA, t-RNA, y-RNA, RNA mimics, DNA, and combinations thereof.

6. The method according to claim 5, wherein said cargo is TGF-beta.

7. The method according to claim 5, wherein said genome editing system is a CRISPR system.

8. The method according to claim 7, wherein said CRISPR system is CRISPR-Cas9 system.

9. The method of claim 5, wherein said microRNA or said siRNA specifically binds to a transcript encoding a mutated or non-mutated oncogene.

10. The method of claim 9, wherein said oncogene is KRAS G12D, KRAS G12C, KRASG12V, N-Myc, c-Myc, or L-Myc.

11. The method according to claim 1, wherein said membrane vesicles have at least one physiological property different from the population of extracellular vesicles or organelles from which said membrane vesicles derive, wherein the physiological property is related to one or more of: biodistribution, cellular uptake, half-life, pharmacodynamics, potency, dosing, immune response, loading efficiency, stability, or reactivity to other compounds.

12. The method according to claim 11, wherein said different physiological property is improved targeting efficiency, improved delivery, or an increase in therapeutic cargo to a recipient cell, organ, or subject.

13. The method according to claim 12, wherein said cargo is loaded into said membrane vesicles more efficiently than said cargo is loaded into extracellular vesicles or organelles from which said membrane vesicles are derived.

14. The method according to claim 1, wherein said extracellular vesicles are a sub-population of extracellular vesicles derived from an extracellular vesicle bulk, or wherein said organelles are one sub-type of organelle derived from a plurality of organelles.

15. The method according to claim 14, further comprising prior to step a:
contacting an epitope specific binder with said extracellular vesicle bulk or said organelles; and
separating said sub-population of extracellular vesicles or sub-type of organelles from said extracellular vesicle bulk or plurality of organelles.

16. The method according to claim 15, wherein said epitope specific binder is an antibody, phage or an aptamer.

17. The method according to claim 16, wherein said epitope specific binder is an antibody against at least one mitochondrial membrane protein.

18. The method according to claim 16, wherein said epitope specific binder is an antibody against the surface marker CD63.

19. Therapeutic membrane vesicles comprising:
vesicles formed from membranes, said membranes being derived from extracellular vesicles or organelles, wherein said membrane vesicles are loaded with a therapeutic cargo.

20.-41. (canceled)

42. A method of separating a sub-population of extracellular vesicles from an extracellular vesicle bulk, comprising
contacting an epitope specific binder with said extracellular vesicle bulk; and
separating said sub-population of extracellular vesicles from said extracellular vesicle bulk.

43.-48. (canceled)
